(19) United States
(12) Patent Application Publication
(10) Pub. No.: US 2025/0195916 A1
(43) Pub. Date: Jun. 19, 2025

(54) SYSTEMS AND METHODS FOR CONTROLLING PHOTOACTIVATABLE PHARMACEUTICAL AGENTS

(71) Applicant: Eli Lilly and Company, Indianapolis, IN (US)

(72) Inventors: Hans Chiwuike AJIEREN, New Caney, TX (US); Pedro IRAZOQUI, Baltimore, MD (US)

(21) Appl. No.: 18/703,453

(22) PCT Filed: Oct. 14, 2022
(86) PCT No.: PCT/US2022/046639

§ 371 Date: Apr. 22, 2024

Related U.S. Application Data
(60) Provisional application No. 63/270,213, filed on Oct. 21, 2021.

(51) Int. Cl. A61N 5/06

(52) U.S. Cl. CPC A61N 5/062; A61N 5/0601; A61N 2005/063; A61N 2005/0651; A61N 2005/0663; A61N 2005/0667

(57) ABSTRACT

A system for controlling a photoactivatable pharmaceutical agent in a subject includes a photomodulator and a controller. The photomodulator is operable to emit a first wavelength of light and a second wavelength of light. The first wavelength of light is configured to activate the photoactivatable pharmaceutical agent, and the second wavelength of light is configured to deactivate the photoactivatable pharmaceutical agent. The controller is operably coupled to the photomodulator. The controller is operable to cause the photomodulator to (1) emit the first wavelength of light and thereby activate the photoactivatable pharmaceutical agent, and (2) emit the second wavelength of light and thereby deactivate the photoactivatable pharmaceutical agent.

### FIELD OF THE DISCLOSURE

The present disclosure relates to systems and methods for activating and deactivating pharmaceutical agents in the body of a subject. More specifically, the present disclosure relates to photomodulator systems and methods for controlling photoactivatable pharmaceutical agents in the body of a subject.

### BACKGROUND OF THE DISCLOSURE

Treatment of various localized medical conditions, such as some forms of cancer and some skin conditions, typically involves generally (that is, non-locally) providing one or more pharmaceutical agents to a subject. Such treatments are typically used because locally providing one or more pharmaceutical agents is infeasible and/or ineffective. Such pharmaceutical agents, despite effectively treating diseased tissue, also often negatively affect healthy tissue. Accordingly, improved systems and methods for providing treatments via pharmaceutical agents are needed.

### SUMMARY OF THE DISCLOSURE

The present disclosure provides photomodulator systems and methods for controlling one or more photoactivatable pharmaceutical agents in the body of a subject.

According to an embodiment of the present disclosure, a system for controlling a photoactivatable pharmaceutical agent in a subject includes a photomodulator and a controller. The photomodulator is operable to emit a first wavelength of light and a second wavelength of light. The first wavelength of light is configured to activate the photoactivatable pharmaceutical agent, and the second wavelength of light is configured to deactivate the photoactivatable pharmaceutical agent. The controller is operably coupled to the photomodulator, and the controller is operable to cause the photomodulator to (1) emit the first wavelength of light and thereby activate the photoactivatable pharmaceutical agent, and (2) emit the second wavelength of light and thereby deactivate the photoactivatable pharmaceutical agent.

In some embodiments, activation is modulated by a physiological event (which may also be referred to as an “outer closed-loop”). In some embodiments, activation is modulated by a measured level of active compound in the body (which may also be referred to as an “inner closed-loop”).

In some embodiments, the photomodulator includes a first emitter configured to emit the first wavelength of light and a second emitter configured to emit the second wavelength of light.

In some embodiments, the photomodulator includes a plurality of first emitters configured to emit the first wavelength of light and a plurality of second emitters configured to emit the second wavelength of light.

In some embodiments, the plurality of second emitters is arranged to define at least a portion of a perimeter, and the plurality of first emitters is disposed within the portion of the perimeter.

In some embodiments, the system further includes a wearable device configured to be secured to the skin of the subject, and the wearable device includes the photomodulator.

In some embodiments, the wearable device includes the controller.

In some embodiments, the system further includes an implantable device configured to be disposed within the body of the subject, and the implantable device includes the photomodulator.

In some embodiments, the implantable device includes the controller.

In some embodiments, the system further includes an implantable device having the photomodulator and configured to be disposed within the body of the subject. The implantable device includes an activating probe and a deactivating cuff. The activating probe is configured to be disposed in a targeted mass of the subject, and the activating probe includes a first emitter configured to emit the first wavelength of light. The deactivating cuff is configured to be secured to a blood vessel coupled to the targeted mass of the subject, and the deactivating cuff includes a second emitter configured to emit the second wavelength of light.

In some embodiments, the system further includes an optical element coupled to the photomodulator.

In some embodiments, the optical element is a microlens array.

In some embodiments, the microlens array includes an electrochromic coating.

In some embodiments, the optical element is a diffractive optical element.

In some embodiments, the controller is operable to cause the photomodulator to simultaneously emit the first wavelength of light and the second wavelength of light.

According to another embodiment of the present disclosure, a system for controlling a photoactivatable pharmaceutical agent in a subject includes a photomodulator, an activation sensor, and a controller. The photomodulator is operable to emit light, and the light is configured to activate the photoactivatable pharmaceutical agent. The activation sensor (which may be, for example, part of an “inner closed-loop”) is operable to determine an amount of the photoactivatable pharmaceutical agent activated in the subject. The controller is operably coupled to the photomodulator and the activation sensor. The controller is operable to cause the photomodulator to emit the light and thereby activate the photoactivatable pharmaceutical agent, and modify an amount of the light emitted by the photomodulator in response to the amount of the photoactivatable pharmaceutical agent activated in the subject determined by the activation sensor.

According to another embodiment of the present disclosure, a system for controlling a photoactivatable pharmaceutical agent in a subject includes a photomodulator, an activation sensor, and a controller. The photomodulator is operable to emit light, and the light is configured to activate the photoactivatable pharmaceutical agent. The activation sensor (which may be, for example, part of an “outer closed-loop) is operable to determine a physiological event warranting activation of the pharmaceutical agent in the subject. The controller is operably coupled to the photomodulator and the activation sensor. The controller is operable to cause the photomodulator to emit the light and thereby activate the photoactivatable pharmaceutical agent, and modify an amount of the light emitted by the photomodulator in response to changes in the physiological state of the patient.

According to another embodiment, both inner and outer closed-loops are implemented in the same system or device.

In some embodiments, the activation sensor includes an excitation emitter, a fluorophore, and a fluorescence detector. The excitation emitter is operable to emit a first wavelength of light. The fluorophore is configured to emit a second wavelength of light upon receiving the first wavelength of light when the fluorophore is in the presence of an activated photoactivatable pharmaceutical agent. The fluorescence detector is operable to detect the second wavelength of light emitted by the fluorophore.

In some embodiments, the activation sensor further includes a waveguide optically coupling the excitation emitter to the fluorophore.

In some embodiments, the waveguide includes an optical fiber.

In some embodiments, the fluorescence detector includes a photodiode.

In some embodiments, the excitation emitter includes a light emitting diode.

In some embodiments, the system further includes an excitation filter optically coupling the excitation emitter to the fluorophore.

In some embodiments, the system further includes an emission filter optically coupling the fluorophore to the fluorescence detector.

In some embodiments, the activation sensor includes a chemosensor.

In some embodiments, the activation sensor includes a plurality of chemosensors.

In some embodiments, the activation sensor further includes an excitation emitter operable to emit a first wavelength of light, and the plurality of chemosensors each include a fluorophore configured to emit a second wavelength of light upon receiving the first wavelength of light when the fluorophore is in the presence of activated photoactivatable pharmaceutical agent. The activation sensor further includes a fluorescence detector operable to detect the second wavelength of light emitted by the fluorophores.

In some embodiments, the activation sensor further includes a plurality of ring resonators operable to filter the second wavelength of light emitted by the fluorophores.

In some embodiments, the activation sensor further includes a waveguide optically coupling the excitation emitter to the plurality of chemosensors.

In some embodiments, the activation sensor further includes a waveguide optically coupling the plurality of chemosensors to the fluorescence detector.

In some embodiments, the waveguide includes an optical fiber.

In some embodiments, the activation sensor is a first activation sensor, the system further includes a second activation sensor operable to determine a physiological event in the subject, and the controller is further operable to modify an amount of the light emitted by the photomodulator in response to the second activation sensor determining the physiological event in the subject.

### BRIEF DESCRIPTION OF THE DRAWINGS

The above-mentioned and other advantages and objects of this invention, and the manner of attaining them, will become more apparent, and the invention itself will be better understood, by reference to the following description of embodiments of the invention taken in conjunction with the accompanying drawings, wherein:

FIG. 1 is a schematic representation of a system for controlling a photoactivatable pharmaceutical agent in a subject, according to an embodiment of the present disclosure.

FIG. 2 is a perspective view of a wearable device for controlling a photoactivatable pharmaceutical agent in a subject, according to another embodiment of the present disclosure.

FIG. 3 is an exploded perspective view of the wearable device of FIG. 2.

FIG. 4 is a perspective view of a wearable device for controlling a photoactivatable pharmaceutical agent in a subject, according to another embodiment of the present disclosure.

FIG. 5 is an exploded perspective view of the wearable device of FIG. 4.

FIG. 6 is a top view of an exemplary diffractive optical element for devices for controlling a photoactivatable pharmaceutical agent in a subject, according to an embodiment of the present disclosure.

FIG. 7 is an enlarged top view of a portion of the diffractive optical element of FIG. 6.

FIG. 8 is a top view of another exemplary diffractive optical element for devices for controlling a photoactivatable pharmaceutical agent in a subject, according to an embodiment of the present disclosure.

FIG. 9 is an enlarged top view of a portion of the diffractive optical element of FIG. 8.

FIG. 10 is a top view of yet another exemplary diffractive optical element for devices for controlling a photoactivatable pharmaceutical agent in a subject, according to an embodiment of the present disclosure.

FIG. 11 is a perspective view of a further exemplary diffractive optical element for devices for controlling a photoactivatable pharmaceutical agent in a subject, according to an embodiment of the present disclosure.

FIG. 12 is a side view of an exemplary microlens array for devices for controlling a photoactivatable pharmaceutical agent in a subject, according to an embodiment of the present disclosure.

FIG. 13 is a top view of a wearable device for controlling a photoactivatable pharmaceutical agent in a subject, according to yet another embodiment of the present disclosure.

FIG. 14 is a side view of the wearable device of FIG. 13.

FIG. 15 is a perspective view of an implantable device for controlling a photoactivatable pharmaceutical agent in a subject, according to an embodiment of the present disclosure.

FIG. 16 is a perspective view of an implantable device for controlling a photoactivatable pharmaceutical agent in a subject, according to another embodiment of the present disclosure.

FIG. 17 is an enlarged perspective view of the implantable device of FIG. 16.

FIG. 18 is a schematic representation of a sensor for sensing an activated photoactivatable pharmaceutical agent in a subject, according to an embodiment of the present disclosure.

FIG. 19 is a schematic representation of an electronics assembly for use with the sensor of FIG. 18.

FIG. 20 is a perspective sectional view of a sensor for sensing an activated photoactivatable pharmaceutical agent in a subject, according to another embodiment of the present disclosure.

FIG. 21 is a schematic representation of a sensor for sensing an activated photoactivatable pharmaceutical agent in a subject, according to another embodiment of the present disclosure.

FIG. 22 is a graph illustrating a spectrum of chemosensors of the sensor of FIG. 21.

FIG. 23 is a graph illustrating spectra of treatment area ring resonators and boundary ring resonators of the sensor of FIG. 21.

FIG. 24 is a graph illustrating a spectrum of fluoresced light transmitted to a waveguide of the sensor of FIG. 21.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present invention.

### DETAILED DESCRIPTION OF THE DRAWINGS

Systems and methods according to embodiments of the present disclosure facilitate controlling one or more photoactivatable pharmaceutical agents, which may also be referred to as photoactivatable medications or drugs. Generally, photoactivatable pharmaceutical agents may be configured to an active state, or activated, by being exposed to one or more wavelengths of light. In the active state, photoactivatable pharmaceutical agents are configured to cause one or more pharmacological effects to the body of the subject. Stated another way, in the active state photoactivatable pharmaceutical agents are configured to provide pharmacological treatment to the subject. Photoactivatable pharmaceutical agents may also be configured to an inactive state, or deactivated, by being exposed to one or more different wavelengths of light. In the inactive state, photoactivatable pharmaceutical agents do not cause pharmacological effects to the body of the subject. Stated another way, in the inactive state photoactivatable pharmaceutical agents are inert relative to the body of the subject.

Photoactivatable pharmaceutical agents may include, for example, photocaged raseglurant, more specifically photocaged raseglurant as described by J. Font et al., “Optical control of pain in vivo with a photoactive mGlu5 receptor negative allosteric modulator,” eLife, vol. 6, p. e23545, April 2017, doi: 10.7554/eLife.23545, or photocaged morphine, more specifically photocaged morphine as described by M. López-Cano et al., “Remote local photoactivation of morphine produces analgesia without opioid-related adverse effects,” British Journal of Pharmacology, p. bph. 15645, September 2021, doi: 10.1111/bph.15645. Both of the aforementioned publications are incorporated by reference herein.

Photoactivatable pharmaceutical agents may initially be administered to a subject, for example, in liquid form and/or via syringe assemblies or intravenous (“IV”) conduits. The photoactivatable pharmaceutical agents may then be distributed in the body of the subject via the circulatory system.

Systems and methods according to embodiments of the present disclosure facilitate activating and deactivating photoactivatable pharmaceutical agents at different bodily locations and/or certain times. Stated another way, systems and methods according to embodiments of the present disclosure facilitate controlling photoactivatable pharmaceutical agents with spatial/temporal specificity.

Systems according to embodiments of the present disclosure are operated in a manner generally as described herein by a user (for example, a healthcare professional, a caregiver, or another person) to control one or more photoactivatable pharmaceutical agents in a subject (for example, another person or the user).

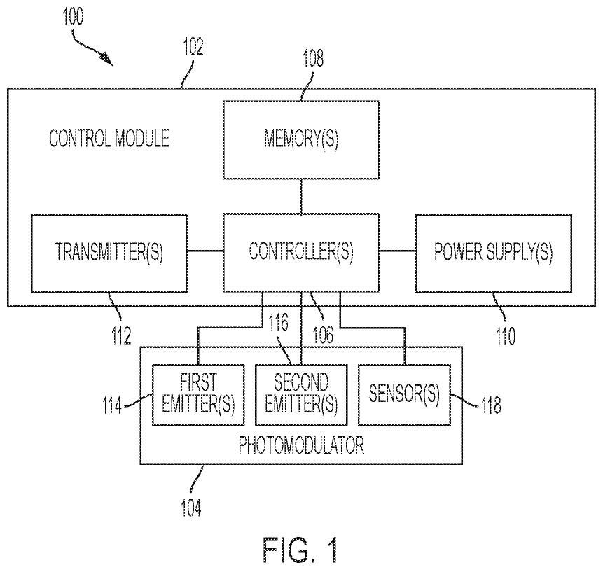

FIG. 1 schematically illustrates a system 100 for controlling one or more photoactivatable pharmaceutical agents in a subject according to an embodiment of the present disclosure. Generally, the system 100 includes a control module 102 that is operably coupled to one or more photomodulators 104 (illustratively, via wireless communication—as used in the present application, the term “operably coupled” includes wired data communication and wireless data communication, whether direct or indirect via one or more intervening devices or components, and such data communication may be continuous or intermittent). The control module 102 is operable to cause the photomodulator 104 to (1) emit one or more wavelengths of light that activate the photoactivatable pharmaceutical agent, and (2) emit one or more different wavelengths of light that deactivate the photoactivatable pharmaceutical agent. These aspects are described in further detail below.

With continued reference to FIG. 1, the control module 102 includes one or more controllers 106, which may be any device or component capable of executing stored software and/or firmware code that, when executed by the controller 106, causes the system 100 to perform the functions described herein. The controller 106 may be, for example, an application-specific integrated circuit (ASICs), a field-programmable gate array (FPGA), a digital signal processor (DSP), hardwired logic, combinations thereof, or the like.

The controller 106 operably couples to a memory 108 (illustratively, via wired communication) for storing, for example, software or firmware code or sensed parameters, as described in further detail below. The memory 108 may be any suitable computer readable medium that is accessible by the controller 106. The memory 108 may be a single storage device or multiple storage devices, may be located internally or externally to the controller 106, and may include both volatile and non-volatile media. The memory 108 may be, for example, a random-access memory (RAM), a read-only memory (ROM), an electrically erasable programmable ROM (EEPROM), a flash memory, a magnetic storage device, an optical disk storage, or any other suitable medium which is capable of storing data and which is accessible by the controller 106.

The controller 106 also operably couples to a power supply 110 (illustratively, via wired communication) for providing power to the various components of the system 100. The power supply 110 may be, for example, one or more rechargeable batteries, one or more inductive/wireless power receivers, or the like.

The controller 106 further operably couples to a transmitter 112 (illustratively, via wired communication) for wirelessly transmitting information, such as sensed parameters (which may be, for example, sensed as part of an “outer closed-loop”), to one or more remote devices (not shown), such as mobile devices, including smartphones, smartwatches, or tablet devices, personal computers, remote computers or databases, or the like. The transmitter 112 may be, for example, a Bluetooth transmitter, an IEEE 802.11 transmitter, a cellular communication transmitter, a near-field communication transmitter, or the like. The transmitter 112 may be continuously coupled or intermittently coupled to the remote device. A transceiver (not shown) may be used instead of the transmitter 112 to facilitate providing information from a remote device to the system 100. Such information may include, for example, software updates.

With continued reference to FIG. 1, the photomodulator 104 includes one or more first emitters 114 and one or more second emitters 116. The first emitters 114 emit a first wavelength of light that activates a photoactivatable pharmaceutical agent. More specifically, the first emitters 114 may be configured to emit light having a wavelength of, for example, about 450 nm, or blue light. Illustratively, the first emitters 114 may only emit the first wavelength of light. Alternatively, the first emitters 114 may be capable of emitting multiple wavelengths of light, and the controller 106 may cause the first emitters 114 to emit the first wavelength of light. The first emitters 114 may be light-emitting diodes (“LEDs”) or the like. Similarly, the second emitters 116 emit a second wavelength of light that deactivates a photoactivatable pharmaceutical agent. More specifically, the second emitters 116 may be configured to emit light having a wavelength of, for example, about 550 nm, or green light. Illustratively, the second emitters 116 may only emit the second wavelength of light. Alternatively, the second emitters 116 may be capable of emitting multiple wavelengths of light, and the controller 106 may cause the second emitters 116 to emit the second wavelength of light. The second emitters 116 may be LEDs or the like.

Alternatively, instead of including first emitters 114 and second emitters 116 as described above, the photomodulator 104 may include one or more emitters that are configured to emit the first wavelength of light (for example, at certain times and/or based on sensed parameters) and the second wavelength of light (for example, at other times and/or based on other sensed parameters).

With further reference to FIG. 1, the photomodulator 104 also includes one or more sensors 118. The sensors 118 may sense physiological parameters and/or parameters associated with a photoactivatable pharmaceutical agent. Physiological parameters may include, for example, physiological parameters indicative of pain experienced by the subject, physiological parameters indicative of inflammation (such as cytokine levels), a temperature of the subject and/or of a body part of the subject, physiological parameters indicative of diabetic events (such as changes to glucose levels), heart rate changes, and characteristics of a targeted mass (for example, diseased tissue). Parameters associated with a photoactivatable pharmaceutical agent may include, for example, the presence or an amount of inactivated photoactivatable pharmaceutical agent and/or the presence or an amount of activated photoactivatable pharmaceutical agent. As described in further detail below, the controller 106 may modify operation of the system 100, more specifically an amount of light emitted by the photomodulator 104, based on parameters sensed by the sensor 118.

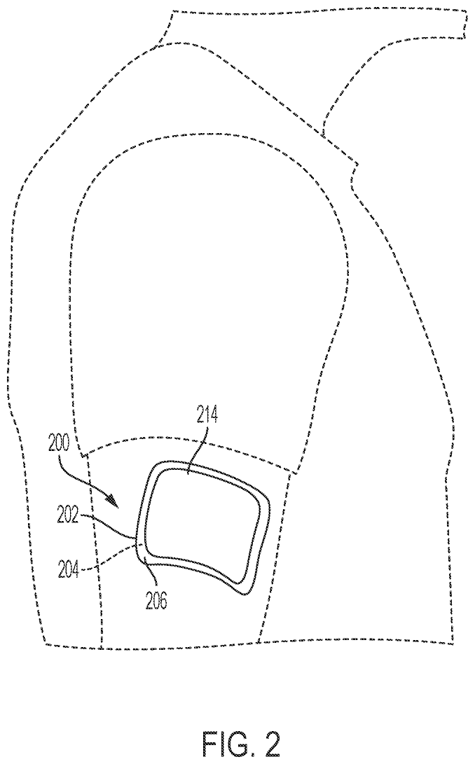

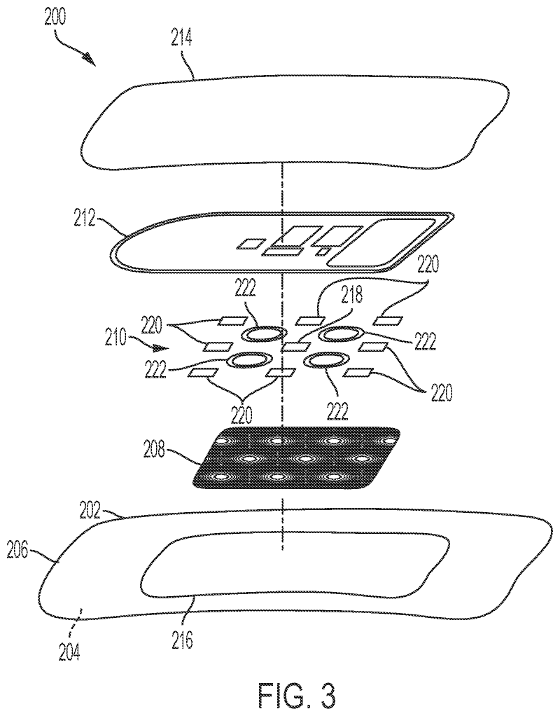

Systems according to the present disclosure may be incorporated into devices that are configured to be worn by a subject, or wearable devices. Referring now to FIGS. 2 and 3, a wearable device 200 according to an embodiment of the present disclosure is illustrated. The wearable device 200 is a more specific embodiment of the system 100 described above. Accordingly, the wearable device 200 includes some of the same components and operates in a similar manner to the system 100 described above. Generally, the wearable device 200 is constructed in the form of a patch. More specifically, the wearable device 200 includes a flexible base or housing 202 that is configured to be detachably secured to the skin of a subject (for example, via one or more adhesives—not shown). A subject-facing side 204 of the base 202 is configured to be secured to the skin of the subject. An opposite-facing side 206 of the base 202 carries an optical element 208 (FIG. 3), a photomodulator 210 (FIG. 3), a control module 212 (FIG. 3), and a flexible cover 214 for securing these components to the base 202. The base 202 may further include a transparent portion 216 (FIG. 3) adjacent to the optical element 208 and the photomodulator 210 to facilitate emitting light from the wearable device 200.

With specific reference to FIG. 3, the control module 212 may be the same or similar to the control module 102 described above. Similarly, the photomodulator 210 may be the same or similar to the photomodulator 104 described above. More specifically, the photomodulator 210 includes one or more first emitters 218 that are configured to emit the first wavelength of light and one or more second emitters 220 that are configured to emit the second wavelength of light. The photomodulator 210 also includes a plurality of sensors 222. The optical element 208 is configured to receive light from the first emitters 218 and the second emitters 220 and more uniformly direct the light toward the subject. The optical element 208 may take various forms, and exemplary details are described below.

With continued reference to FIG. 3, the first emitters 218 and the second emitters 220 are arranged in a grid, more specifically a three-row, three-column grid. Illustratively, a first emitter 218 is disposed at the center of the grid, and second emitters 220 form a perimeter of the grid. Accordingly, the wearable device 200 may be positioned such that the first emitter 218 is adjacent to a targeted mass of the subject (for example, diseased tissue). The first emitter 218 may emit the first wavelength of light to activate a photoactivatable pharmaceutical agent in the targeted mass, and the second emitters 220 may simultaneously emit the second wavelength of light to deactivate the photoactivatable pharmaceutical agent as it circulates away from the targeted mass. In other embodiments, the first emitters 218 and the second emitters 220 may have different arrangements.

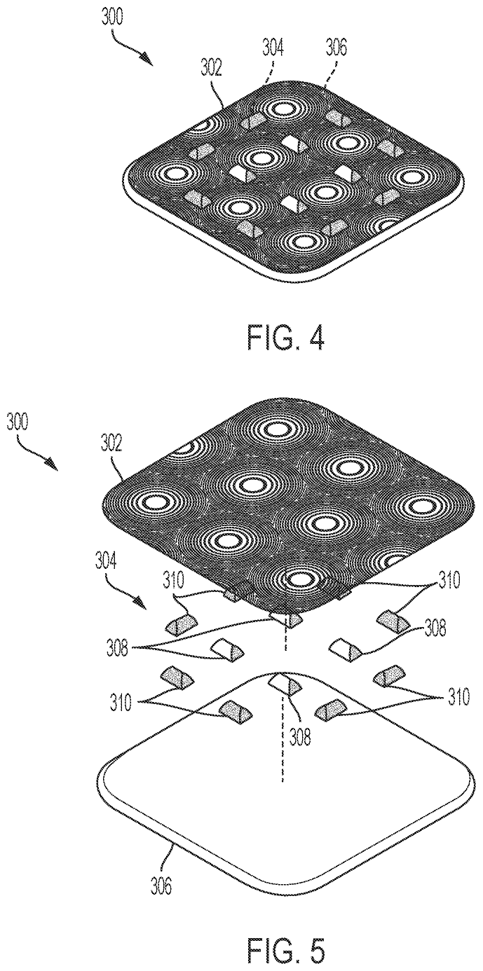

Referring now to FIGS. 4 and 5, a wearable device 300 according to another embodiment of the present disclosure is illustrated. The wearable device 300 is similar to the wearable device 200 described above. More specifically, the wearable device 300 includes a base (not shown) that carries an optical element 302, a photomodulator 304, a control module (not shown), and a cover 306 for securing these components to the base. Referring specifically to FIG. 5, first emitters 308 and second emitters 310 of the photomodulator 304 are generally arranged in the shape of a plus symbol (that is “+”). Illustratively, first emitters 308 are disposed at the sides of the symbol, and second emitters 310 form a perimeter of the symbol. Accordingly, the wearable device 300 may be positioned such that the first emitters 308 are adjacent to a targeted mass of the subject. The first emitters 308 may emit the first wavelength of light to activate a photoactivatable pharmaceutical agent in the targeted mass, and the second emitters 310 may simultaneously emit the second wavelength of light to deactivate the photoactivatable pharmaceutical agent as it circulates away from the targeted mass.

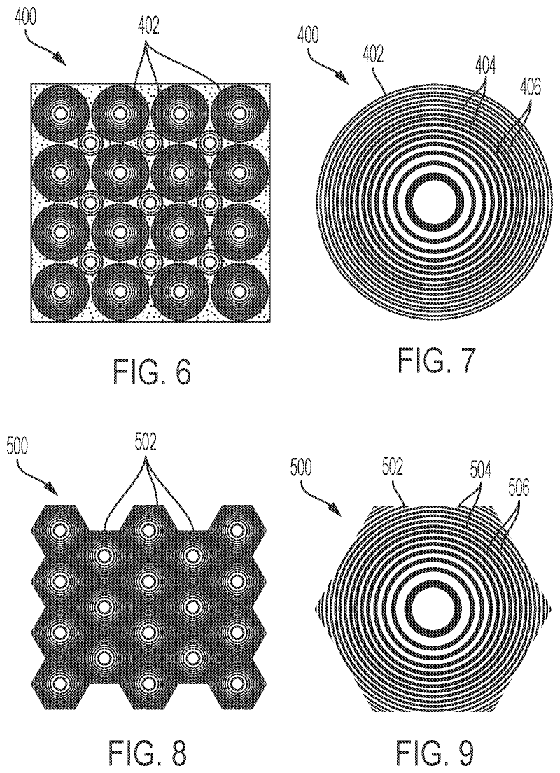

As described briefly above, devices according to the present disclosure may include optical elements for directing light toward a subject. Such optical elements may take various forms. For example, the optical elements may include lenses or diffractive optical elements, elements including patterns of opaque and transparent portions. FIGS. 6 and 7 illustrate an exemplary diffractive optical element 400. The diffractive optical element 400 includes a grid of circular zone plates 402 (three of which are identified in FIG. 6), and each circular zone plate 402 includes alternating and concentric opaque rings 404 and transparent rings 406 (two of each being identified in FIG. 7). Illustratively, the rings 404, 406 decrease in thickness proceeding away from the center of each circular zone plate 402. The thickness of the rings 404, 406 may also be adjusted to create a desired optical wavefront at a targeted mass through diffraction. Factors such as the position of the light source, the position of the targeted mass, and the wavelength of light being diffracted influence the final thickness selected for the zone plate rings. The circular zone plates 402 may be provided in different sizes, with FIG. 6 illustrating two such sizes.

FIGS. 8 and 9 illustrate another exemplary diffractive optical element 500. The diffractive optical element 500 includes a grid of hexagonal shapes 502 (three of which are identified in FIG. 8), and each hexagonal shape 502 includes alternating and concentric opaque rings 504 and translucent rings 506 (two of each being identified in FIG. 9). Illustratively, the rings 504, 506 are truncated near the edge of each hexagonal shape 502, and the rings 504, 506 decrease in thickness proceeding away from the center of each hexagonal shape 502.

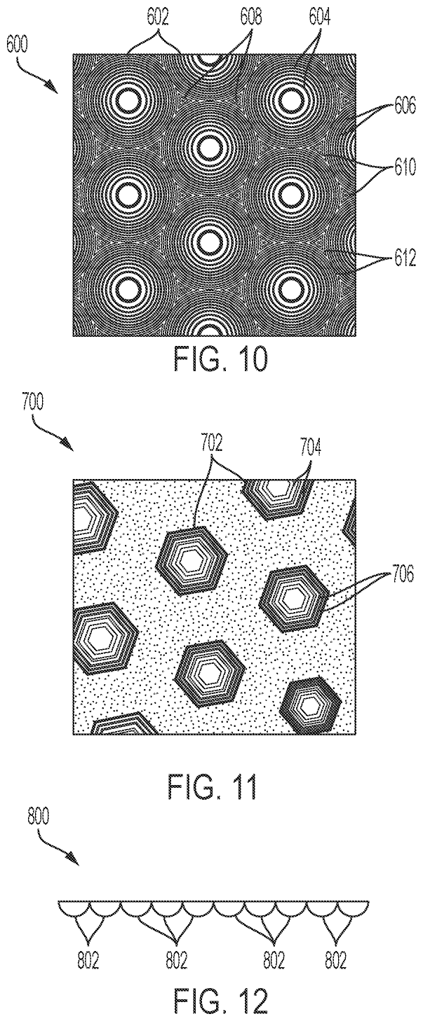

FIG. 10 illustrate another exemplary diffractive optical element 600. The diffractive optical element 600 includes a grid of circular shapes 602 (two of which are identified in FIG. 10), and each circular shape 602 includes alternating and concentric opaque rings 604 and translucent rings 606 (two of each being identified in FIG. 10). Illustratively, the rings 604, 606 decrease in thickness proceeding away from the center of each circular shape 602. The diffractive optical element 600 also includes generally triangular shapes 608 between adjacent circular shapes 602 (two of which are identified in FIG. 10). Each triangular shape 608 includes alternating and concentric opaque triangles 610 and translucent triangles 612 (two of each being identified in FIG. 10). Illustratively, the triangles 610, 612 decrease in thickness proceeding away from the center of each triangular shape 608.

FIG. 11 illustrates yet another exemplary diffractive optical element 700. The diffractive optical element 700 includes a grid of hexagonal shapes 702 (two of which are identified in FIG. 11), and each hexagonal shape 702 includes alternating and concentric opaque hexagons 704 and translucent hexagons 706 (two of each being identified in FIG. 11). Illustratively, the hexagons 704, 706 decrease in thickness proceeding away from the center of each hexagonal shape 702.

As another example, the optical elements may be microlens arrays, or arrays including a plurality of relatively small lenses. Such arrays may more uniformly direct light toward a subject and may be relatively compact compared to standard lenses. FIG. 12 provides a profile view (that is, a view from the side) of an exemplary microlens array 800. The array 800 includes a plurality of relatively small lenses 802, and the lenses 802 may be arranged in a grid. More specifically, the lenses 802 may be arranged horizontally, as illustrated, and extending into the page. The microlens array 800 may be constructed of one or more flexible, biocompatible materials. The lenses 802 may have an electrochromic coating (not shown), and each lens 802 may be controlled in a similar manner to a pixel. As a result, the microlens array 800 may facilitate emitting the first wavelength of light and/or the second wavelength on relatively small areas of the body of the subject.

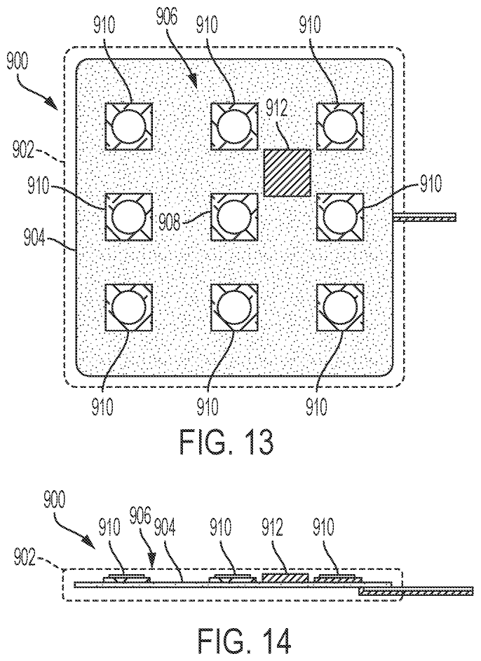

Wearable devices according to the present disclosure may be modified in various other manners. For example, one or more components of the system 100 may be remotely disposed from wearable devices according to the present disclosure. As a more specific example, FIGS. 13 and 14 illustrate a wearable device 900 according to another embodiment of the present disclosure is illustrated. The wearable device 900 is similar to the wearable devices described above. More specifically, the wearable device 900 includes a housing 902 that carries a flexible substrate 904, and the flexible substrate 904 carries a photomodulator 906. The photomodulator 906 includes one or more first emitters 908 and one or more second emitters 910. The flexible substrate 904 also carries a sensor 912. The photomodulator 906 and the sensor 912 operably couple to a control module (not shown) via wired communication. Alternatively, the photomodulator 906 and the sensor 912 operably couple to the control module via wireless communication. The control module may be part of another wearable device (not shown) or a non-wearable device (not shown).

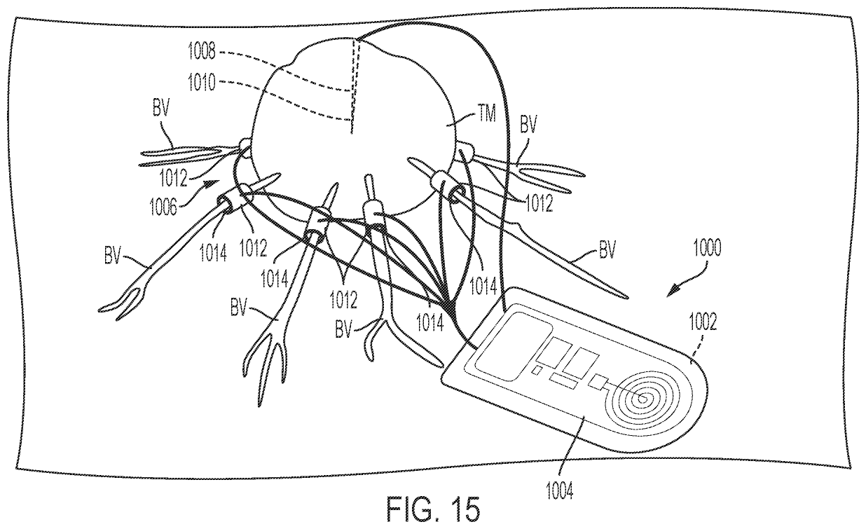

Systems according to the present disclosure may be incorporated into devices that are configured to be implanted in a subject, or implantable devices. Referring now to FIG. 15, an implantable device 1000 according to an embodiment of the present disclosure is illustrated. The implantable device 1000 is a more specific embodiment of the system 100 described above. Accordingly, the implantable device 1000 includes some of the same components and operates in a similar manner to the system 100 described above. Generally, the implantable device 1000 includes a housing 1002 that is configured to be implanted in a subject. The housing 1002 carries a control module 1004, and the control module 1004 operably couples to a photomodulator 1006 via wired communication. The control module 1004 may be the same or similar to the control module 102 described above. The photomodulator 1006 includes an activating probe 1008 that is configured to be disposed in a targeted mass TM. The probe 1008 has one or more first emitters 1010 that are configured to emit the first wavelength of light to the targeted tissue TM. The photomodulator 1006 also includes a plurality of deactivating cuffs 1012 that are configured to extend around, or otherwise be secured to, blood vessels BV coupled to the targeted mass TM. Each cuff 1012 includes one or more second emitters 1014 that are configured to emit the second wavelength of light. Accordingly, the first emitters 1010 may emit the first wavelength of light to activate a photoactivatable pharmaceutical agent in the targeted mass TM, and the second emitters 1014 may emit the second wavelength of light to deactivate the photoactivatable pharmaceutical agent as it moves away from the targeted mass TM and through the blood vessels BV.

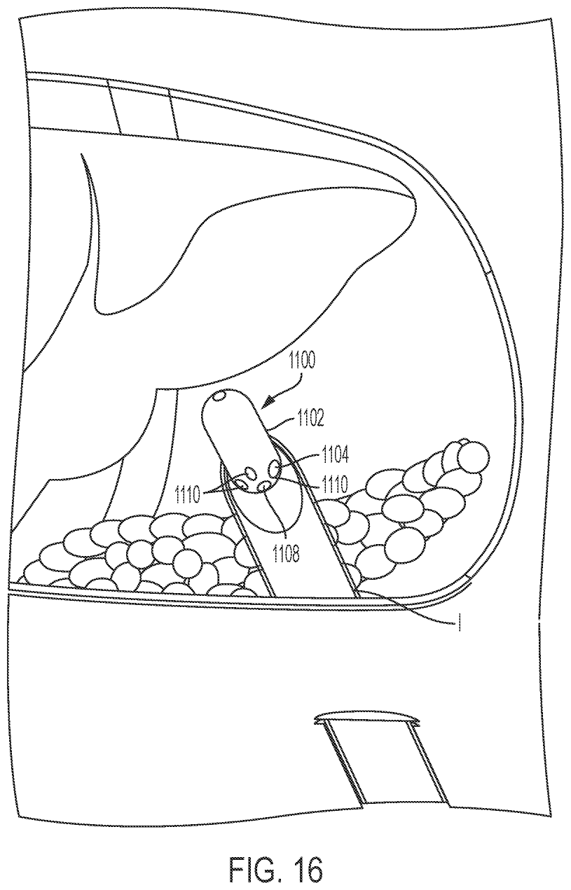

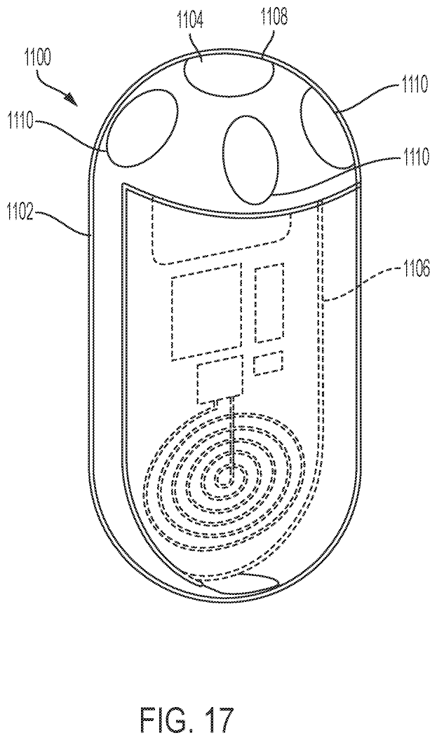

Referring now to FIGS. 16 and 17, an implantable device 1100 according to another embodiment of the present disclosure is illustrated. The implantable device 1100 is a more specific embodiment of the system 100 described above. Accordingly, the implantable device 1100 includes some of the same components and operates in a similar manner to the system 100 described above. Generally, the implantable device 1100 is constructed in the form of a capsule, and the implantable device 1100 may be implanted percutaneously via an introducer I. More specifically, the implantable device 1100 includes a housing 1102 that is configured to be implanted in a subject. The housing 1102 carries a photomodulator 1104 and a control module 1106 (FIG. 17). The control module 1106 may be the same or similar to the control module 102 described above. Similarly, the photomodulator 1104 may be the same or similar to the photomodulator 104 described above. More specifically, the photomodulator 1104 includes one or more first emitters 1108 that are configured to emit the first wavelength of light and one or more second emitters 1110 that are configured to emit the second wavelength of light. The first emitters 1108 and the second emitters 1110 may emit light simultaneously or at different times, or the implantable device 1100 may emit the first wavelength of light and a second, similar implantable device (not shown) may emit the second wavelength of light. Alternatively, in some embodiments, the first emitters 1108 and the second emitters 1110 may be the same type of emitter and may collectively be configured to emit the first wavelength of light at a first time and to emit the second wavelength of light at a second, different time. In some embodiments, the implantable device 1100 includes one or more sensors (such as the sensors 118—shown elsewhere) for sensing physiological parameters and/or parameters associated with a photoactivatable pharmaceutical agent. The sensors may be disposed adjacent to the first emitters 1108 and the second emitters 1110 or other locations on or within the implantable device 1100.

As described briefly above, operation of the system 100 may be modified based on parameters sensed by the sensors 118. More specifically, the system 100 may modify an amount of light emitted by the photomodulator 104 based on parameters sensed by the sensors 118. Modifying the amount of light emitted by the photomodulator 104 may include, for example, modifying the intensity of the emitted light, modifying the duration of light emission, modifying a pattern of light emission (for example, varying timing of light being emitted and not being omitted), modifying the size of an area on which light is emitted (even if the intensity per unit area remains constant).

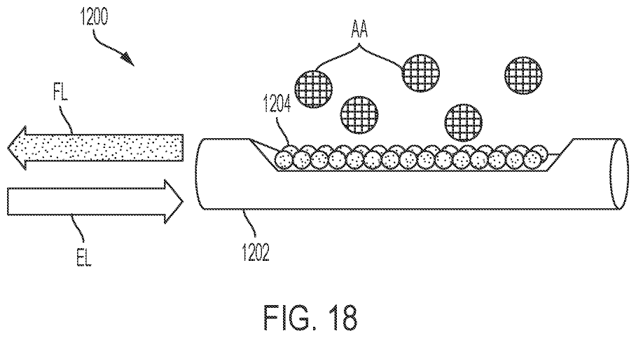

The sensors 118 may take various forms. For example, one or more of the sensors 118 may sense parameters associated with a photoactivatable pharmaceutical agent. FIG. 18 schematically illustrates an exemplary activation sensor 1200 for sensing such parameters. More specifically, the activation sensor 1200 is configured to sense the presence and an amount of an activated photoactivatable pharmaceutical agent in a subject. The sensor 1200 is configured to be disposed internally relative to the subject.

Generally, the sensor 1200 includes a waveguide 1202, such as an optical fiber, for transmitting a first wavelength of light EL (which may be referred to as “excitation light”—having, for example, a wavelength of about 475 nm) to a fluorophore 1204. The fluorophore 1204 emits a second wavelength of light FL (which may be referred to as “fluoresced light”—having, for example, a wavelength of about 515 nm) upon receiving the first wavelength of light EL and in the presence of an activated photoactivatable pharmaceutical agent AA (as determined based on the activated agent itself or a biomarker indicative of the activated agent, such as calcium ions). The fluorophore 1204 emits an amount the second wavelength of light FL corresponding to the present amount of the activated photoactivatable pharmaceutical agent AA. The waveguide 1202 transmits the second wavelength of light FL emitted by the fluorophore 1204 back to a sensor that detects and/or measures an amount of the second wavelength of light FL, and the system 100 may modify an amount of light emitted by the photomodulator 104 (FIG. 1) based on the amount of the second wavelength of light FL transmitted by the waveguide 1202.

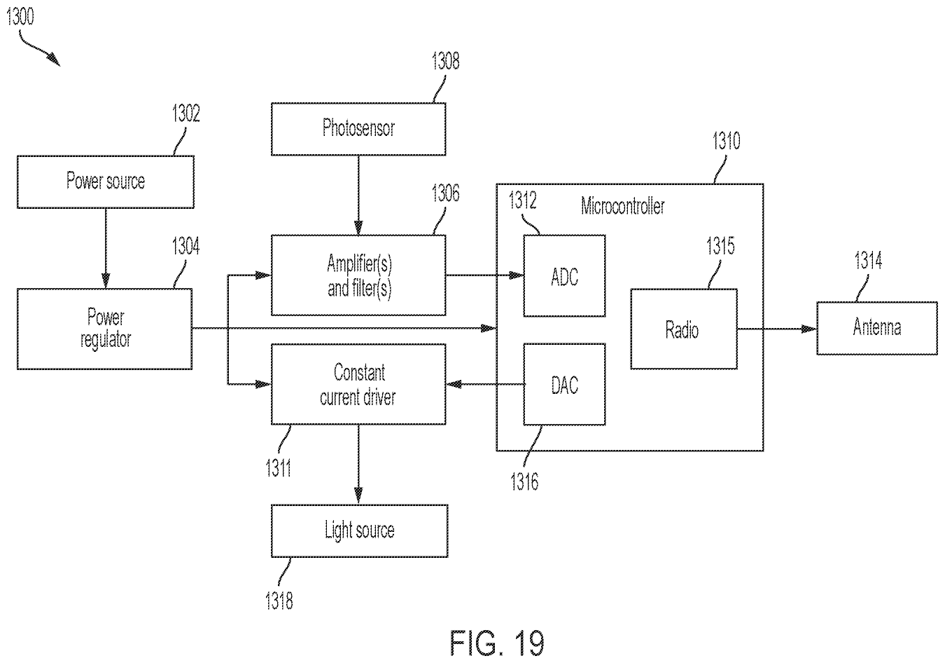

FIG. 19 schematically illustrates an exemplary electronics assembly 1300 for use with the activation sensor 1200 (FIG. 18). The electronics assembly 1300 includes a power source or supply 1302 for providing power to the various components of the electronics assembly 1300. The power supply 1302 may be, for example, the same power supply 110 described above or a different device. The power supply 1302 operably couples to a power regulator 1304 (illustratively, via wired communication), which includes one or more low-dropout regulators (not shown). The power regulator 1304 operably couples to one or more amplifiers and filters 1306, a controller or microcontroller 1310, and a constant current driver 1311 (illustratively, via wired communication). The amplifiers and filters 1306 operable couple to a fluorescence detector or photosensor 1308 (illustratively, via wired communication). The controller 1310 may be, for example, the same controller 106 described above or a different device. The controller 1310 operably couples to the amplifiers and filters 1306 via an analog-to-digital converter 1312 (illustratively, via wired communication). The controller 1310 also operably couples to a transmitter or antenna 1314, via a radio 1315. The controller 1310 also operably couples to the constant current driver 1311 via a digital-to-analog converter 1316 (illustratively, via wired communication). The constant current source converter 1311 operably couples (illustratively, via wired communication) to an excitation emitter or light source 1318. The excitation emitter 1318 emits excitation light to the waveguide 1202 of the sensor 1200 (FIG. 18), and the waveguide 1202 transmits fluoresced light to the fluorescence detector 1308. The controller 1310 detects the fluoresced light received by the fluorescence detector 1308 to determine the presence and the amount of an activated photoactivatable pharmaceutical agent in a subject, and the system 100 may thereby modify an amount of light emitted by the photomodulator 104 (FIG. 1).

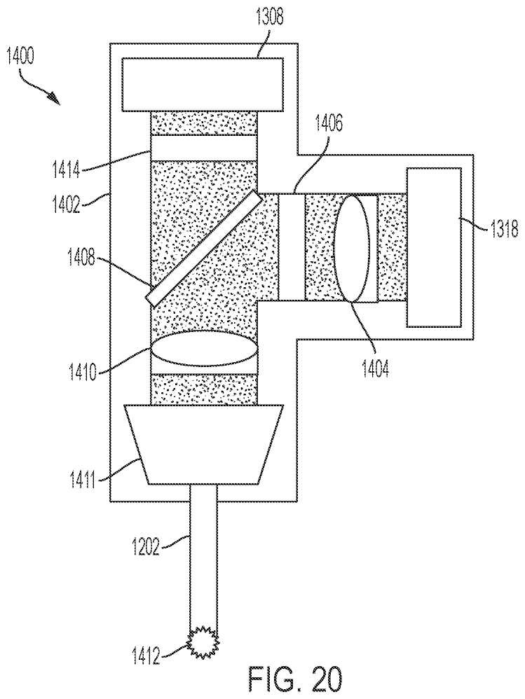

Referring now to FIG. 20, a sensor 1400 according to an embodiment of the present disclosure is illustrated. The sensor 1400 is a more specific embodiment of the sensor 1200 described above. Accordingly, the sensor 1400 includes some of the same components and operates in a similar manner to the sensor 1200 described above. The sensor 1400 may also be used together with the electronics assembly 1300 (FIG. 19). The sensor 1400 includes a housing 1402 that carries various components, including the excitation emitter 1318 of the electronics assembly 1300. The excitation emitter 1318 transmits excitation light to the waveguide 1202 (FIG. 18) via a first lens 1404, an excitation filter 1406, a beam splitter 1408, and a second lens 1410. The waveguide 1202 is carried by a mechanical coupler 1411. The waveguide 1202 transmits the excitation light to an optrode 1412 including the fluorophore 1204 (FIG. 18). Upon receiving the excitation light and in the presence of an activated photoactivatable pharmaceutical agent, as described above, the fluorophore 1204 emits fluoresced light to the waveguide 1202. The waveguide 1202 transmits the fluoresced light to the fluorescence detector 1308 via the second lens 1410, the beam splitter 1408, and an emission filter 1414. The controller 1310 (FIG. 19) detects the fluoresced light received by the fluorescence detector 1308 to determine the presence and the amount of the activated photoactivatable pharmaceutical agent in a subject, and the system 100 may thereby modify an amount of light emitted by the photomodulator 104 (FIG. 1).

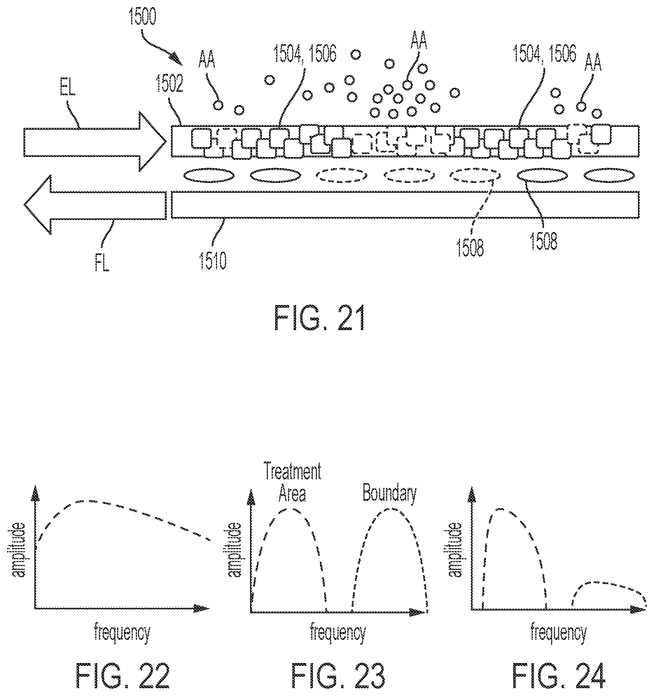

FIG. 21 schematically illustrates another exemplary activation sensor 1500 for sensing parameters associated with a photoactivatable pharmaceutical agent. The activator sensor 1500 may be used in place of, or in addition to, the sensor 1200 (FIG. 18). The activation sensor 1500 is configured to sense the presence, an amount, and a location of an activated photoactivatable pharmaceutical agent in a subject. The sensor 1500 is configured to be disposed internally relative to the subject. Generally, the sensor 1500 includes a first waveguide 1502, such as an optical fiber, for transmitting excitation light EL to one or more chemosensors 1504 (two of which are identified). Each chemosensor 1504 includes a fluorophore 1506 that emits fluoresced light upon receiving the excitation light EL and in the presence of an activated photoactivatable pharmaceutical agent AA (as determined based on the activated agent itself or a biomarker indicative of the activated agent, such as calcium ions). Each fluorophore 1506 emits an amount of fluoresced light corresponding to an adjacent amount of the activated photoactivatable pharmaceutical agent AA. That is, the fluorophores 1506 are configured to independently emit varying amounts the fluoresced light. The fluoresced light emitted by the fluorophores 1506 is filtered by a plurality of ring resonators 1508 (two of which are identified). The ring resonators 1508 depicted in phantom lines indicate “treatment area” ring resonators configured to be disposed adjacent a targeted treatment area of the subject, while the ring resonators 1508 depicted in solid lines depict “boundary” ring resonators configured to be disposed along a boundary or a periphery of the targeted treatment area. Each ring resonator filters light emitted by the fluorophores 1506 to pass through light having certain frequencies and to filter out light having other frequencies. The filtered fluoresced light is received by a second waveguide 1510, such as an optical fiber. The second waveguide 1510 transmits the filtered fluoresced light FL, and the system 100 may modify an amount of light emitted by the photomodulator 104 (FIG. 1) based on the amount of fluoresced light FL and/or location information encoded in the fluoresced light FL.

FIG. 22 illustrates the spectrum of the chemosensors 1504 (FIG. 21). As can be seen, each chemosensor emits light of multiple frequencies. FIG. 23 illustrates the transmission spectra (that is, the amount of light transmitted and not filtered out) of the “treatment area” ring resonators 1508 (FIG. 21-that is, the ring resonators 1508 depicted in phantom lines) and the “boundary” ring resonators 1508 (that is, the ring resonators 1508 depicted in solid lines). FIG. 24 illustrates the spectrum of fluoresced light from the chemosensors 1504 after being filtered through ring resonators 1508 and transmitted to the second waveguide 1510 (FIG. 21). As can be seen in FIG. 24, the filtered light can be expected to occupy two frequency domains—a first frequency domain that comprises light filtered through the “treatment area” ring resonators 1508, and a second frequency domain that comprises light filtered through the “boundary” ring resonators 1508—in this embodiment, the first frequency domain is at a lower frequency than the second frequency domain, but the activation sensor 1500 may be alternatively configured so that the first frequency domain is at a higher frequency than the second frequency domain. By measuring the intensity of the filtered light transmitted to the second waveguide 1510 at each of these different frequency domains, a measurement system may separately determine (i) an amount of light emitted by chemosensors adjacent a treatment area and (ii) an amount of light emitted by chemosensors disposed along a boundary or a periphery of the targeted treatment area. In this way, the measurement system may separately determine an amount of the activated photoactivatable pharmaceutical agent AA at the treatment area as well as in the boundary area.

While this invention has been shown and described as having preferred designs, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

1. A system for controlling a photoactivatable pharmaceutical agent in a subject, the system comprising:
a photomodulator operable to emit a first wavelength of light and a second wavelength of light, the first wavelength of light configured to activate the photoactivatable pharmaceutical agent, and the second wavelength of light configured to deactivate the photoactivatable pharmaceutical agent; and
a controller operably coupled to the photomodulator;
wherein the controller is operable to cause the photomodulator to (1) emit the first wavelength of light and thereby activate the photoactivatable pharmaceutical agent, and (2) emit the second wavelength of light and thereby deactivate the photoactivatable pharmaceutical agent.

2. The system of claim 1, wherein the photomodulator comprises a first emitter configured to emit the first wavelength of light and a second emitter configured to emit the second wavelength of light.

3. The system of claim 1, wherein the photomodulator comprises a plurality of first emitters configured to emit the first wavelength of light and a plurality of second emitters configured to emit the second wavelength of light.

4. The system of claim 3, wherein the plurality of second emitters is arranged to define at least a portion of a perimeter, and the plurality of first emitters is disposed within the portion of the perimeter.

5. The system of claim 1, further comprising a wearable device configured to be secured to a skin of the subject, the wearable device including the photomodulator.

6. (canceled)

7. The system of claim 1, further comprising an implantable device configured to be disposed within a body of the subject, the implantable device including the photomodulator.

8. (canceled)

9. The system of claim 1, further comprising an implantable device including the photomodulator and configured to be disposed within a body of the subject, the implantable device comprising:
an activating probe configured to be disposed in a targeted mass of the subject, the activating probe comprising a first emitter configured to emit the first wavelength of light; and
a deactivating cuff configured to be secured to a blood vessel coupled to the targeted mass of the subject, the deactivating cuff comprising a second emitter configured to emit the second wavelength of light.

10. The system of claim 1, further comprising a microlens array coupled to the photomodulator.

11. (canceled)

12. The system of claim 10, wherein the microlens array comprises an electrochromic coating.

13. The system of claim 1, further comprising a diffractive optical element coupled to the photomodulator.

14. The system of claim 1, wherein the controller is operable to cause the photomodulator to simultaneously emit the first wavelength of light and the second wavelength of light.

15. A system for controlling a photoactivatable pharmaceutical agent in a subject, the system comprising:
a photomodulator operable to emit light, the light configured to activate the photoactivatable pharmaceutical agent; and
an activation sensor operable to determine an amount of the photoactivatable pharmaceutical agent activated in the subject;
a controller operably coupled to the photomodulator and the activation sensor;
wherein the controller is operable to cause the photomodulator to emit the light and thereby activate the photoactivatable pharmaceutical agent, and modify an amount of the light emitted by the photomodulator in response to the amount of the photoactivatable pharmaceutical agent activated in the subject determined by the activation sensor.

16. The system of claim 15, wherein the activation sensor comprises:
an excitation emitter operable to emit a first wavelength of light;
a fluorophore configured to emit a second wavelength of light upon receiving the first wavelength of light when the fluorophore is in the presence of activated photoactivatable pharmaceutical agent; and
a fluorescence detector operable to detect the second wavelength of light emitted by the fluorophore.

17. The system of claim 16, wherein the activation sensor further comprises a waveguide optically coupling the excitation emitter to the fluorophore.

18. (canceled)

19. The system of claim 16, wherein the fluorescence detector comprises a photodiode.

20. The system of claim 16, wherein the excitation emitter comprises a light emitting diode.

21. (canceled)

22. (canceled)

23. The system of claim 15, wherein the activation sensor comprises a chemosensor.

24. (canceled)

25. The system of claim 15, wherein the activation sensor comprises a plurality of chemosensors and an excitation emitter operable to emit a first wavelength of light, and the plurality of chemosensors each comprise a fluorophore configured to emit a second wavelength of light upon receiving the first wavelength of light when the fluorophore is in the presence of activated photoactivatable pharmaceutical agent, and the activation sensor further comprises a fluorescence detector operable to detect the second wavelength of light emitted by the fluorophores.

26. The system of claim 25, wherein the activation sensor further comprises a plurality of ring resonators operable to filter the second wavelength of light emitted by the fluorophores.

27. The system of claim 25, wherein the activation sensor further comprises a waveguide optically coupling the excitation emitter to the plurality of chemosensors.

28. The system of claim 25, wherein the activation sensor further comprises a waveguide optically coupling the plurality of chemosensors to the fluorescence detector.

29. (canceled)

30. The system of claim 15, wherein the activation sensor is a first activation sensor, further comprising a second activation sensor operable to determine a physiological parameter in the subject, and wherein the controller is further operable to modify an amount of the light emitted by the photomodulator in response to the second activation sensor determining the physiological parameter in the subject.
